(19) United States
(12) Patent Application Publication
(10) Pub. No.: US 2004/0106162 A1
(43) Pub. Date: Jun. 3, 2004

(54) Food pathogen sensor using molecularly imprinted polymers

(76) Inventors: Jay A. Glasel, Farmington, CT (US); Carl J. Freeman, Hampden, ME (US)

Correspondence Address: PATRICK R. SCANLON, PIERCE ATWOOD, ONE MONUMENT SQUARE, PORTLAND, ME, 04101, US

(21) Appl. No.: 10/652,972

(22) Filed: Aug. 28, 2003

Related U.S. Application Data
(60) Provisional application No. 60/406,411, filed on Aug. 28, 2002.

(51) Int. Cl. G01N 33/554; G01N 33/569; C12M 1/34

(52) U.S. Cl. 435/7.32; 435/287.2

(57) ABSTRACT

A sensor for sensing pathogens includes a molecularly imprinted polymer membrane applied to a surface of a flexible backing. The molecularly imprinted polymer membrane is capable of selectively binding pathogens such that binding sites will be fluorescent in the absence of pathogens and fluorescence is quenched in the presence of pathogens. Monitoring the fluorescence quenching will provide detection of contamination.

This application claims the benefit of U.S. Provisional Application No. 60/406,411, filed Aug. 28, 2002.

### BACKGROUND OF THE INVENTION

This invention relates generally to detecting pathogens in foods and more particularly to an organic polymer-based rapid food pathogen detector that can be mass-produced inexpensively.

The present vulnerability of civilian and military populations to lethal pathogenic contamination of their food supplies is widely recognized. This vulnerability extends from the sites of origin of food materials to their points of consumption. Providing adequate protection for these populations throughout all the steps from origins to consumption has no easy solutions. An important contribution can be made by developing inexpensive, rapid-throughput methods that allow monitoring of contamination in packaged consumables, and spot testing of raw, unpackaged foodstuffs. To be effective, the testing method must be simple, stable, sensitive, accurate, and reproducible.

In the increasing number of incidents of deliberate contamination of commercial foodstuffs in the United States, detection and identification of the pathogens has always taken place after distribution of the uncontaminated food from production sites to stores and after consumption and resulting sickness. Unless detection methods change, this situation is unlikely to improve, since test methods available to food microbiologists require extensive and time-consuming laboratory workups by highly trained personnel that are impractical to use after food distribution.

While a wide range of biosensor techniques have been previously applied to the food contamination problem, relatively few pathogens can be reliably measured by commercially available equipment. Moreover, available detectors of packaged food contamination incorporate one or more biomolecule-derived elements. These biomolecule-containing devices suffer from the expense of isolation and production of biomolecules and the biomolecule's susceptibility to destruction in the field by dehydration, bacterial action, moderately high temperatures, and other environmental conditions.

Accordingly, there is a need for food pathogen sensors that do not rely on biomolecules.

### SUMMARY OF THE INVENTION

The present invention provides an improved sensor that combines development of synthetic polymers that selectively bind food pathogens with fluorescence excitation/detection technology. A unique aspect of this sensor is the method of specifically binding pathogens. The invention utilizes molecularly imprinted polymers (MIPs) bonded to flexible backings to form thin strips or sheets. These sensor strips selectively bind intact pathogens on the basis of their components' characteristic three-dimensional “footprints” that are permanently imprinted on the polymeric surfaces. The MIP binding sites are fluorescent in the absence of pathogens. When a pathogen is specifically bound to its MIP site, the fluorescence from that site is quenched.

In a version designed for packaged foods, initially sterile sensor strips are inserted in the food packages during manufacture so as to be in contact with food surfaces while the food is in the package. They absorb individual pathogenic species that may develop in, or may be introduced into, the food package. The presence of pathogens in each package is monitored by scanning its sensor strip with an automated external fluorescence scanner that detects the patterns and magnitudes of fluorescence quenching to yield readouts of contamination. For spot testing, sterile sensor strips can be manually applied to the food sample, and detect contamination when scanned with a portable fluorescence scanner.

Previously developed food pathogen detectors have depended on the use of biological macromolecules such as antibodies as the detection agents. In the present invention, MIPs replace biomolecules as the sensor components to which pathogens selectively and strongly bind. The MIPs contain copolymerized fluorophores whose fluorescence emission is suppressed (“quenched”) when pathogens are bound, but is unaffected in the absence of strong binding.

The use of MIPs instead of biomolecules greatly reduces production costs of the final food pathogen detection system while at the same time increases the system's stability in the face of environmental challenges.

The presence of pathogens in food packaged with the present invention is automatically monitored using an external fluorescence scanner as the sensor strip-containing packages are rapidly passed by the fixed sensor. Alternatively, prepackaged sterile sensor strips can be employed for spot testing of food samples using a hand-held version of the scanner. Field-operable biosensors that can alert to food contamination by known pathogens are another alternative.

The present invention and its advantages over the prior art will be more readily understood upon reading the following detailed description and the appended claims with reference to the accompanying drawings.

### DESCRIPTION OF THE DRAWINGS

The subject matter that is regarded as the invention is particularly pointed out and distinctly claimed in the concluding part of the specification. The invention, however, may be best understood by reference to the following description taken in conjunction with the accompanying drawing figures in which:

FIG. 1 is a schematic diagram of one embodiment of a food pathogen sensor.

FIG. 2 illustrates the step of arranging monomers, a fluorophore, and a template pathogen for the preparation of a molecularly imprinted polymer membrane.

FIG. 3 illustrates the step of extracting the template pathogen in the preparation of a molecularly imprinted polymer membrane.

FIG. 4 shows a pathogen bound to an imprinted site of a molecularly imprinted polymer membrane.

FIG. 5 is a schematic diagram of a molecularly imprinted polymer membrane illustrating pathogen binding.

FIG. 6 shows a food package having a food pathogen sensor enclosed in its packaging.

### DETAILED DESCRIPTION OF THE INVENTION

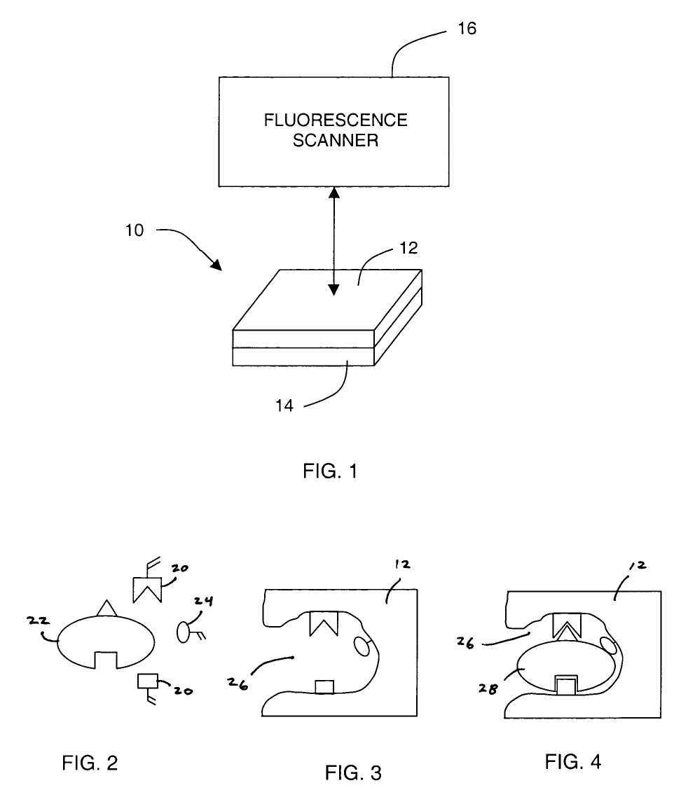

Referring to the drawings wherein identical reference numerals denote the same elements throughout the various views, FIG. 1 shows one embodiment of a food pathogen sensor 10. The sensor 10 includes a membrane 12 of a molecularly imprinted polymer (MIP) applied to a sheet or strip of a flexible backing material 14. The MIP membrane 12 is imprinted with the negative “image” of the three-dimensional structure of at least one pathogen in a manner described below in more detail. In the embodiment of FIG. 1, a fluorescence scanner 16 is provided for scanning the sensor 10 to detect patterns and magnitudes of fluorescence quenching and thereby yield readouts of contamination.

In the field of molecular analysis, the development and use of molecularly imprinted adsorbents has recently undergone a rapidly expanding role (Andersson, L. I. (2000) Molecular imprinting: developments and applications in the analytical chemistry field J Chromatogr B Biomed Sci Appl 745 3-13; and Andersson, L. I. (2000) Molecular imprinting for drug bioanalysis. A review on the application of imprinted polymers to solid-phase extraction and binding assay J Chromatogr B Biomed Sci Appl 739 163-73) and includes the development of MIPs membranes of the type used with the present invention (Takeuchi, T. and Haginaka, J. (1999) Separation and sensing based on molecular recognition using molecularly imprinted polymers J Chromatogr B Biomed Sci Appl 728 1-20). The binding forces between pathogens and MIPs are comparable to those between pathogens and comparably selective antibodies (Vlatakis, G., et al. (1993) Drug assay using antibody mimics made by molecular imprinting Nature 361 645-7). Since binding pathogens to polymers in MIP production does not depend on their biological activity, the task of making these imprinted polymers is simplified by the use of pathogens inactivated by radiation.

The MIPs are preferably synthetic polymers obtained by polymerizing monomers with a cross-linker in the presence of a template pathogen. Referring now to FIGS. 2 and 3, the preparation of a fluorescent MIP membrane 12 first involves arranging monomers 20 with a template organism or molecule 22 of the pathogen of interest (FIG. 2). A fluorophore 24 is also included. Next is the polymerization of the functional and crosslinking monomers 20 in the presence of the template 22 and fluorophore 24. After polymerization, the template 22 is removed by washing or other means, leaving imprinted sites 26 embedded in the solid, highly cross-linked polymer network forming the membrane 12 (FIG. 3). The sites 26 are capable of selectively binding the pathogen. When exposed to the same environmental pathogen, the imprinted sites 26 bind the pathogen 28 non-covalently (FIG. 4). Close proximity of bound pathogen 28 to the fluorescent label quenches the fluorescence by resonant energy transfer. The principle behind the binding of the agent to its MIP is the same as for antigen-antibody binding: the three dimensional shape of the target is recognized with high selectivity by the complementary shape of the binding site, and the target is captured and bound by the actions of short-range intermolecular forces acting between the target and its MIP. Contrasted with antibody and other protein absorbents, MIPs are more stable under changing environmental conditions. The strength of the forces between targets and MIPs have been shown to be the same order of magnitude as forces between antigens and antibodies.

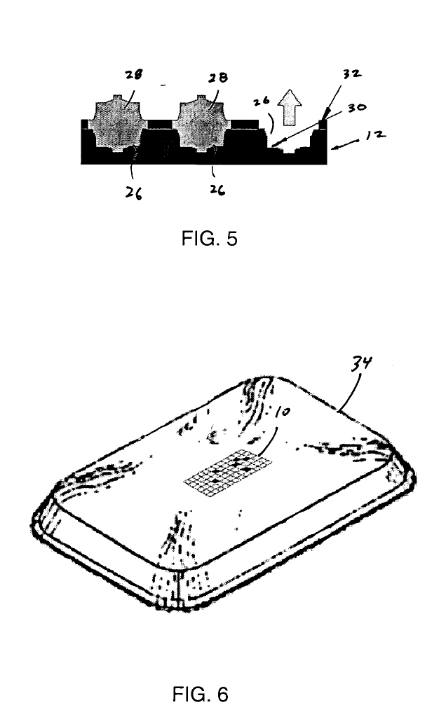

Referring to FIG. 5, a MIP membrane 12 will preferably include a multitude of imprinted sites 26. The fluorescence emission of sites to which a pathogen 28 is bound will be quenched, while the fluorescence emission of sites to which no pathogen is bound will be unaffected. As shown in FIG. 5, the MIP membrane 12 can comprises a fluorescent layer 30 and an anti-fluorescence mask layer 32.

The MIP membranes 12 can contain imprint sites 26 binding to different pathogens. Samples of these membranes 12 that specifically bind different pathogens will be cut into patches and mounted adjacent to one-another to form a sensor strip 10 recognizing two or more pathogens. To increase signal-to-noise ratio in the detection stage, the membrane patches may be attached in different patterns corresponding to each different pathogen-sensitive membrane. The fluorescence scanner 16 can be made to scan the patches in a defined order corresponding to each pattern and accept as positives only those whose quenching follows the same pattern.

In mass production, MIP membranes 12 containing pathogen sensitive patterns may be produced by a variety of methods including production of a master positive stamp which can press (“micromold”) negative images into a polymerizing membrane (Yan, M. and Kapua, A. (2001) Fabrication of molecularly imprinted polymer microstructures Analytica Chimica Acta 435 163-167).

Suitable polymers used for the MIPs include polymers formed as membranes by polymerization of a solution of methacrylic acid and ethylene glycol dimethacrylate monomers on a quartz crystal surface in the presence of the pathogen(s) to be detected.

As mentioned above, the MIP membranes 12 are bonded to flexible backings 14 to form thin strips or sheets. Microporous polypropylene is one suitable backing material.

The detection of pathogens bound to the MIPs will be based on incorporating fluorescent compounds within MIP copolymer membranes. Preparation of MIPs membranes for detecting specific molecules has been described in many publications (Takeuchi, T. and Haginaka, J. (1999) Separation and sensing based on molecular recognition using molecularly imprinted polymers J Chromatogr B Biomed Sci Appl 728 1-20). Details of the quenching of fluorescence emission in MIPs by specific binding of ligands to fluorophore-incorporating MIPs have been published and reviewed (Turkewitsch, P., et al. (1998) Fluorescent functional recognition sites through molecular imprinting. A polymer-based fluorescent chemosensor for aqueous cAMP Analytical Chemistry 70 2025-2030, Ye, L. and Mosbach, K. (2001) Polymers recognizing biomolecules based on a combination of molecular imprinting and proximity scintillation: a new sensor concept J Am Chem Soc 123 2901-2; Takeuchi, T., et al. (2001) Molecularly imprinted polymers with metalloporphyrin-based molecular recognition sites coassembled with methacrylic acid Anal Chem 73 3869-74; and Yano, K. and Karube, I. (1999) Molecularly Imprinted Polymers for Biosensor Applications Trends in Analytical Chemistry 18 199-204). A possible alternative would be to use sensor strips that undergo color changes after contamination. However, fluorescence detection is orders of magnitude more sensitive (Yano, K. and Karube, I. (1999) Molecularly Imprinted Polymers for Biosensor Applications Trends in Analytical Chemistry 18 199-204).

The efficiency of fluorescence quenching is a very short-range effect that depends upon the proximity (R) between the quenching agent (in this case, component of the pathogen binding to the fluorescent MIP) to the fluorophore, and the time the agent spends at that proximity. The distance dependence is approximately 1/R⁶ so that only effective quenching agents are those bound with high affinity at the fluorophore-containing binding site. Since high affinity binding (corresponding to a low dissociation constant; the ratio of “on” to “off” rates for the quenching agent) means that the off-rate is slow, such binding implies relatively long residence times for the quencher at the binding site. None of the work done so far with fluorescent quenching of MIPs has shown non-specific quenching by interfering molecules.

Since high affinity binding is crucial, careful measurement of binding parameters will be part of the development of the sensor strips. It may be necessary to develop MIPs that bind to portions of the pathogens in analogy to the binding of antibodies to portions (coat proteins, carbohydrates, etc.) of infectious particles. This would require biochemical isolation of macromolecular components of pathogens by standard biochemical methods.

FIG. 6 shows a food package 34 with a sensor strip 10 having multiple pathogen sensitive patterns enclosed in the packaging. Generally, the fluorescence scanner 16 (not shown in FIG. 6) emits an excitation beam as it scans the sensor strip 10. The excitation beam scans each pattern separately. Fluorescent emission from each pattern is detected and stored separately for automated analysis, wherein the patterns of quenching identify each pathogen present. The pathogen-sensitive arrays can also contain alignment and emission normalization markers.

Some basic issues in scanning packaged food for contamination are rapidity of scanning and low false positives. The sensor strips will employ identifying array patterns of MIPs that specifically bind selected individual food pathogen species and quench the fluorescence emission in the pattern corresponding to the species bound. These fluorescence emissions properties will be detected by rapidly passing the sensor strip-containing packages on a conveyer belt under an automated fluorescence scanner, or using a handheld version of the scanner on the packages or food samples. Because fluorescence response is effectively instantaneous, very rapid throughput can be achieved. Since MIPs are much more stable than biological entities such as antibodies, the lifetimes of sensor strip-containing packages will be much longer than similar schemes based on antibody or other biomolecular specific pathogen binding.

The present invention provides the many advantages including the following:

able to identify known food pathogens.

does not require expensive biological components (such as antibodies, other proteins, or nucleic acids) for detection as do all currently available food pathogen sensors

The signal detection system that can be used for high-volume screening of packaged foods

can be mass-produced cheaply and incorporated into packaged foods as easily as barcodes

much more resistant to environmental biological and chemical degradation than detectors based on biological components

have a high tolerance to mechanical and thermal stress

have excellent storage stabilities

While specific embodiments of the present invention have been described, it will be apparent to those skilled in the art that various modifications thereto can be made without departing from the spirit and scope of the invention as defined in the appended claims.

What is claimed is:

1. A sensor for sensing food pathogens, said sensor comprising a molecularly imprinted polymer membrane imprinted with one or more binding sites that are capable of selectively binding pathogens such that binding sites will be fluorescent in the absence of pathogens and fluorescence is quenched in the presence of pathogens.

2. The sensor of claim 1 wherein said molecularly imprinted polymer membrane is applied to a flexible backing.

3. The sensor of claim 1 wherein said molecularly imprinted polymer membrane has fluorescent compounds incorporated therein.

4. The sensor of claim 1 wherein said molecularly imprinted polymer membrane includes binding sites that bind different pathogens.

5. A sensor for sensing food pathogens, said sensor comprising:
a flexible backing; and
a molecularly imprinted polymer membrane applied to a surface of said flexible backing, said molecularly imprinted polymer membrane being imprinted with one or more binding sites that are capable of selectively binding pathogens such that binding sites will be fluorescent in the absence of pathogens and fluorescence is quenched in the presence of pathogens.

6. The sensor of claim 5 wherein said molecularly imprinted polymer membrane has fluorescent compounds incorporated therein.

7. The sensor of claim 5 wherein said molecularly imprinted polymer membrane includes binding sites that bind different pathogens.

8. A system for sensing food pathogens, said system comprising:
a molecularly imprinted polymer membrane imprinted with one or more binding sites that are capable of selectively binding pathogens such that binding sites will be fluorescent in the absence of pathogens and fluorescence is quenched in the presence of pathogens; and
a fluorescence scanner for scanning said molecularly imprinted polymer membrane to detect patterns and magnitudes of fluorescence quenching.

9. The sensor of claim 8 wherein said molecularly imprinted polymer membrane is applied to a flexible backing.

10. The sensor of claim 8 wherein said molecularly imprinted polymer membrane has fluorescent compounds incorporated therein.

11. The sensor of claim 8 wherein said molecularly imprinted polymer membrane includes binding sites that bind different pathogens.
